(19) United States
(12) Patent Application Publication
(10) Pub. No.: US 2025/0171699 A1
(43) Pub. Date: May 29, 2025

(54) PROCESS FOR MAXIMIZING LIGHT OLEFINS VIA AROMATICS SATURATION IN FCC AND STEAM CRACKER BASED CRUDE TO CHEMICALS CONFIGURATION

(71) Applicant: Lummus Technology LLC, Houston, TX (US)

(72) Inventors: Sumit Mittal, Houston, TX (US); Shekhar Tewari, Houston, TX (US); Ankur Jain, Houston, TX (US); Vishal Varshney, Houston, TX (US); Rama Rao Marri, Houston, TX (US); Ronald Venner, Houston, TX (US)

(73) Assignee: Lummus Technology LLC, Houston, TX (US)

(21) Appl. No.: 18/957,557

(22) Filed: Nov. 22, 2024

(30) Foreign Application Priority Data
Nov. 24, 2023 (IN) 202321079916

(51) Int. Cl. C10G 69/06; C07C 6/04

(52) U.S. Cl. CPC C10G 69/06; C07C 6/04; C10G 2400/20; C10G 2400/22

(57) ABSTRACT

Systems and processes herein integrate a crude separation unit, a steam cracker unit, a fluid catalytic cracking unit, an aromatics processing unit, and a diprolene pyrolysis gasoline unit for separating a whole crude oil or other wide boiling hydrocarbon mixtures and for producing olefins and/or aromatics. Systems and processes herein provide various schemes to provide flexibility and valorize aromatics produced via steam cracker unit or fluid catalytic cracking unit by directly extracting and/or converting them into cycloalkanes and feeding them to a steam cracker unit to produce more valuable olefins.

### FIELD OF THE DISCLOSURE

Embodiments of the present disclosure generally relate to systems and processes for the production of light olefins, such as ethylene and propylene from direct crude processing. More particularly, embodiments herein are directed toward processes and systems for maximizing the production of light olefins by saturating the aromatics generated in the process.

### BACKGROUND

In recent years, the changing dynamics of the market have prompted refineries to explore new opportunities in petrochemical production from crude oil, shifting their focus beyond fuel production as fuel demand is depleting. Among the key building blocks for the petrochemical industry, ethylene and propylene hold significant importance. Due to the depleting demand of the fuels and increasing demand of the petrochemicals, refiners are being compelled to find the options to maximize petrochemicals from processing a variety of crude oils. Ethylene and propylene are expected to be in high demand, as they are the primary feed stock for the petrochemical/polymer industries.

### SUMMARY OF THE CLAIMED EMBODIMENTS

Embodiments herein are directed toward processes and systems for maximizing light olefins via aromatics saturation in fluid catalytic cracking (FCC) and steam cracker based crude to chemicals configurations.

In one aspect, embodiments disclosed herein relate to a process for producing olefins and aromatics from wide boiling hydrocarbon feedstock. The process includes separating the wide boiling hydrocarbon feedstock into a light fraction and a heavy fraction. The heavy fraction is catalytically cracked to produce a catalytically cracked effluent, which is fractionated to recover at least a wet gas fraction, a naphtha range fraction, and a heavy catalytically cracked fraction. The process further includes compressing and separating the wet gas fraction to form a light compressed fraction and a heavy compressed fraction, removing impurities from the light compressed fraction to form a purified light compressed fraction, and stripping the heavy compressed fraction to recover a stripped heavy fraction and a stripper gas fraction. The stripper gas fraction is combined with the wet gas fraction prior to the compressing and separating step. The light fraction is thermally cracked in a steam cracker unit to produce a thermally cracked effluent, which is then quenched and separated to recover a fuel oil fraction and a light thermally cracked fraction. The light thermally cracked fraction is mixed with the purified light compressed fraction to form a combined catalytic and thermally cracked light fraction, which is dried to form a dried lights fraction, followed by separating the dried lights fraction to recover a C4+ hydrocarbon fraction and a C3− hydrocarbon fraction. The C4+ fraction is divided into a one or more C5− fractions and a C6+ fraction, followed by mixing the C6+ fraction with the stripped heavy fraction to form a combined C6+ fraction, which is partially hydrogenated to form a hydrogenated C6+ fraction. The hydrogenated C6+ fraction is then separated to recover an offgas fraction, an aromatics rich naphtha fraction, and a raffinate fraction; and the C3− fraction is separated to recover a hydrogen-containing offgas fraction, an ethylene fraction, a propylene fraction, and a light paraffin fraction. The aromatics rich naphtha fraction is fed to an aromatics processing unit for processing the C6 to C8 fraction in one or more of an aromatics dealkylation unit, an aromatics extraction unit, and an aromatics saturation unit to recover a non-aromatic hydrocarbon stream, and the non-aromatic hydrocarbon stream is fed to the steam cracker unit.

In another aspect, embodiments disclosed herein relate to a system for producing olefins and aromatics from a wide boiling hydrocarbon feedstock. The system includes a separation system for separating the wide boiling hydrocarbon feedstock into a light fraction and a heavy fraction; a fluid catalytic cracking unit for catalytically cracking the heavy fraction to produce a catalytically cracked effluent. A fractionation system is provided for fractionating the catalytically cracked effluent to recover at least a wet gas fraction, a naphtha range fraction, and a heavy catalytically cracked fraction. A wet gas compression system compresses and separates the wet gas fraction to form a light compressed fraction and a heavy compressed fraction. Also provided is an impurities removal system for removing impurities from the light compressed fraction to form a purified light compressed fraction, a stripper for stripping the heavy compressed fraction to recover a stripped heavy fraction and a stripper gas fraction, and a flow line for combining the stripper gas fraction with the wet gas fraction upstream of the wet gas compression system. The system also includes a steam cracker system for thermally cracking the light fraction to produce a thermally cracked effluent, and a quench and separation system for quenching and separating the thermally cracked effluent to recover a fuel oil fraction and a light thermally cracked fraction. A mixer is provided for combining the light thermally cracked fraction with the purified light compressed fraction to form a combined catalytic and thermally cracked light fraction, and a compression and drying system is provided for drying the combined catalytic and thermally cracked light fraction to form a dried lights fraction. Still further, the system includes a lights separation system for separating the dried lights fraction to recover a C4+ hydrocarbon fraction and a C3− hydrocarbon fraction, a splitter for splitting the C4+ fraction into one or more C5− fractions and a C6+ fraction, and a mixer for mixing the C6+ fraction with the stripped heavy fraction to form a combined C6+ fraction. A hydrogenation system is also provided for partially hydrogenating the combined C6+ fraction to form a hydrogenated C6+ fraction. A heavies separation system is fluidly connected for separating the hydrogenated C6+ fraction to recover an offgas fraction, an aromatics rich naphtha fraction, and a raffinate fraction. Additionally, a product recovery section is provided for separating the C3− fraction to recover a hydrogen-containing offgas fraction, an ethylene fraction, a propylene fraction, and a light paraffin fraction. The system also includes an aromatics processing unit for processing the C6 to C8 fraction in one or more of an aromatics dealkylation unit, an aromatics extraction unit, and an aromatics saturation unit to produce and recover a non-aromatic hydrocarbon stream, along with a flow line for feeding the non-aromatic hydrocarbon stream to the steam cracker system.

Other aspects and advantages will be apparent from the following description and the appended claims.

### BRIEF DESCRIPTION OF DRAWINGS

FIG. 1 illustrates a simplified process flow diagram of systems and processes according to one or more embodiments disclosed herein.

FIG. 2 a simplified process flow diagram of systems and processes according to one or more embodiments disclosed herein.

### DETAILED DESCRIPTION

Embodiments herein relate to processes and systems that take crude oil and/or low value heavy hydrocarbons as feed and produce petrochemicals, such as light olefins and diolefins (ethylene, propylene, butadiene, and/or butenes) and aromatics. More specifically, embodiments herein are directed toward methods and systems for making olefins and aromatics by thermal cracking and catalytic cracking, where the effluents from the thermal and catalytic cracking are co-processed for the efficient recovery of ethylene and other valuable products.

Embodiments herein are described with respect to crude oil, such as whole crude oil or a desalted whole crude oil, but any high boiling end point hydrocarbon mixture can be used. Processes disclosed herein can be applied to crudes, condensates and hydrocarbon with a wide boiling curve, including those having end points higher than 500° C. Such hydrocarbon mixtures may include whole crudes, virgin crudes, hydroprocessed crudes, gas oils, vacuum gas oils, heating oils, jet fuels, diesels, kerosenes, gasolines, synthetic naphthas, raffinate reformates, Fischer-Tropsch liquids, Fischer-Tropsch gases, natural gasolines, distillates, virgin naphthas, natural gas condensates, atmospheric pipestill bottoms, vacuum pipestill streams including bottoms, wide boiling range naphtha to gas oil condensates, heavy non-virgin hydrocarbon streams from refineries, vacuum gas oils, heavy gas oils, atmospheric residuum, hydrocracker wax, and Fischer-Tropsch wax, among others. In some embodiments, the hydrocarbon mixture may include hydrocarbons boiling from the naphtha range or lighter to the vacuum gas oil range or heavier.

One or more of the above hydrocarbon feedstocks may be fed to systems and processes herein to produce olefins and aromatics. Systems and processes herein include a separation system for separating the wide boiling hydrocarbon feedstock into a light fraction and a heavy fraction. Separation of the whole crude or other wide boiling hydrocarbon feedstock into the desired light and heavy fractions may be performed using one or more separators (distillation columns, flash drums, etc.).

In some embodiments, separation of the petroleum feeds may be performed in an integrated separation device (ISD), such as disclosed in US20130197283. In the ISD, an initial separation of a low boiling fraction is performed in the ISD based on a combination of centrifugal and cyclonic effects to separate the desired vapor fraction from liquid.

In other embodiments, separation of the petroleum feeds may be performed in a Hot Oil Processing Scheme (HOPS unit), such as described in U.S. Pat. No. 10,793,793, for example. In the HOPS unit, the hydrocarbon feedstock is preheated, mixed with dilution steam, and separated to recover a light fraction, vapor mixed with dilution steam, and a heavy fraction, a liquid stream comprising compounds that cannot be easily vaporized.

The heavy fraction is fed to a fluid catalytic cracking unit for catalytically cracking the heavy fraction to produce a catalytically cracked effluent. FCC units may include riser reactors, regenerators, and catalyst separation systems, and are well known in the art and not described further herein. The catalytically cracked effluent is fed to a fractionation system, such as a main fractionator of a FCC unit. The fractionation system is used for fractionating the catalytically cracked effluent to recover at least a wet gas fraction, a naphtha range fraction, and a heavy catalytically cracked fraction. Separation of the catalytically cracked effluent into the desired fractions may be performed using one or more separators (distillation columns, flash drums, etc.).

The wet gas fraction is then processed to separate lighter and heavier components in the wet gas and to remove various impurities before further processing. A wet gas compression system is provided for compressing and separating the wet gas fraction to form a light compressed fraction and a heavy compressed fraction. The compression system may include one or more compressors, aftercoolers, and flash drums or separators to compress the wet gas and condense heavier components therein, thereby producing a heavy compressed fraction and a light compressed fraction.

The light compressed fraction is then fed to an impurities removal system for removing impurities from the light compressed fraction and to form a purified light compressed fraction. The impurities removal system may include one or more adsorption beds, absorption systems, membrane separators, or other separation systems useful for removing contaminants such as AsH₃, PH₃, Hg, as well as impurities such as carbon dioxide, NOx and oxygen, among others, from the light compressed fraction.

The heavy compressed fraction is fed to a stripper for stabilizing the heavy compressed fraction, removing any dissolved or entrained gases within the heavier hydrocarbon fraction(s) recovered from the compression system. Stripping of the heavy compressed fraction produces a stripped heavy fraction and a stripper gas fraction. The stripper gas fraction may then be combined with the wet gas fraction for co-processing within the wet gas compression system.

A steam cracker system is provided for thermally cracking the light fraction to produce a thermally cracked effluent. If desired, the light fraction may be further separated into two or more fractions of distinct boiling ranges for processing within the steam cracker system at conditions most favorable to the hydrocarbons within the respective fractions. The steam cracker system may include one or more furnaces, each including convective coils for pre-heating the respective feeds and radiant coils for superheating the feeds to cracking temperatures.

The one or more steam cracker effluents are then quenched and separated. For example, the effluents may be collectively or individually quenched to halt the cracking reaction, quickly bringing the temperature of the effluent to below steam cracking temperatures. Quench may be performed in transfer line exchangers, direct or indirect quench against various hydrocarbon or steam streams present in the process, and other heat exchange to reduce a temperature of the steam cracker effluent to desired separation feed temperatures. Following quenching and heat recovery, the thermally cracked effluent may be fractionated to recover a light thermally cracked fraction and a fuel oil fraction.

The light thermally cracked fraction may be mixed with the purified light compressed fraction (from impurities removal) to form a combined catalytic and thermally cracked light fraction. Mixing or combining streams herein, such as the light thermally cracked fraction and the purified light compressed fraction, may be performed, for example, in a simple y- or t-type mixing device, which may or may not include static mixers. Other mixing devices known in the art may also be used for combining streams as described herein.

The combined catalytic and thermally cracked light fraction is then compressed, dried, and separated. A compression and drying system is provided for drying the combined catalytic and thermally cracked light fraction to form a dried lights fraction. The compression system may include one or more single or multi-stage compressors, as well as aftercoolers, to separate water from the steam cracked effluent and to provide a compressed combined light fraction at desired temperatures and pressures for separation of the hydrocarbons therein into desired fractions. The compression and drying system may include one or more of coalescers, adsorbents (e.g., a molecular sieve dryer), membrane separators, or absorption systems useful for separating liquid or vaporous water from the hydrocarbons.

The dried lights fraction is then fed to a lights separation system for separating the dried lights fraction to recover a C4+ hydrocarbon fraction and a C3− hydrocarbon fraction. The lights separation system may include one or more separators (distillation columns, flash drums, etc., as well as associated overheads condensation/reflux systems and reboiler systems) to provide the desired separation into the C4+ hydrocarbon fraction and a C3− hydrocarbon fraction. In some embodiments, a single distillation column is used to separate the dried lights fraction.

The C4+ hydrocarbon fraction, which may include C4, C5, C6, and heavier hydrocarbons contained in the combined catalytic and thermally cracked light fraction, is then fed to a splitter for separating the C4+ fraction into a one or more C5− fractions and a C6+ fraction. The splitter may be, for example, a distillation column, flash drums, etc., as well as associated overheads condensation/reflux systems and reboiler systems to provide the desired separation. The C5− fraction, which may be further separated into a C4 fraction and a C5 fraction, may be hydrogenated and fed to the steam cracker system for continued production of ethylene and propylene. Alternatively, the C4s and C5s may be recovered as a product fraction or fed to catalytic cracking system for continued production of ethylene and propylene.

The C6+ fraction is combined with the stripped heavy fraction to form a combined C6+ fraction. The C6+ fraction may include various olefins, paraffins, and aromatics. The C6+ hydrocarbons are then processed to recover an aromatics rich naphtha fraction. The C6+ fraction may be processed, for example, in a diprolene pyrolysis gasoline unit to partially hydrogenate and separate the C6+ fraction to recover an offgas fraction, the aromatics rich naphtha fraction, and a paraffinic rich raffinate. The diprolene pyrolysis gasoline unit may include, for example, a hydrogenation system for partially hydrogenating the combined C6+ fraction to form a hydrogenated C6+ fraction, and a heavies separation system for separating the hydrogenated C6+ fraction to recover an offgas fraction, an aromatics rich naphtha fraction (C6 to C8 or C6 to C9 hydrocarbons, for example), and a raffinate fraction (C9+ or C10+, for example). A flow line may be provided for feeding the offgas fraction to the compression and drying system for co-processing along with the quenched thermally cracked effluent. A flow line may also be provided for feeding the raffinate fraction to the catalytic cracking reactor section.

The C3− hydrocarbon fraction from the lights separation system is fed to a product recovery section for separating the C3− fraction to recover a hydrogen-containing offgas fraction, an ethylene fraction, a propylene fraction, and one or more light paraffin fractions. The product recovery section may include one or more separators (distillation columns, flash drums, etc., as well as associated overheads condensation/reflux systems and reboiler systems) to provide the desired separations. In some embodiments, the system may include a demethanizer or an absorber-demethanizer, a depropanizer, a deethylenizer, and a depropylenizer, allowing for separate recovery of a methane fraction, an ethane fraction, an ethylene fraction, a propane fraction, and a propylene fraction, among others.

The separation of the C3− hydrocarbon fraction may include one or more feed/effluent exchangers, flash drums, etc., to provide the desired separation temperatures, and in some embodiments any cold box or exchanger used is maintained at non-cryogenic temperatures to avoid the formation of N₂O₃ within the separation system. For example, in some embodiments, the absorber demethanizer and the associated overheads system and cold boxes may be operated at temperatures of greater than −130° C., such as greater than-120° C., greater than −110° C., or greater than −100° C., or, in some embodiments, greater than −90° C., thereby limiting the formation of N₂₀₃. In other embodiments, the absorber demethanizer system may be operated at an overheads temperature of approximately −80° C. or greater or −70° C. or greater in yet other embodiments, such as described in U.S. Pat. No. 9,422,210, for example. Embodiments herein may thus incorporate a warmer-than-typical cold box (typical being from about −130° C. to −160° C., for example) and an absorber demethanizer to mitigate these risks. These additional measures ensure not only the safe operation but also the efficient recovery of ethylene.

The systems and processes herein may provide for continued processing of one or more of the above-noted product streams to increase process flexibility and improve the yield of ethylene, propylene, and other desired products. For example, a flow line may be provided for feeding the naphtha range fraction to either or both of the fluid catalytic cracking unit and the steam cracker system. As another manner to increase the yield of ethylene and propylene, a flow line may be provided for feeding the light paraffin fraction(s) (ethane, propane) from the product recovery section to the steam cracker system. As yet another means, a flow line or flow lines may be provided for feeding a portion of the one or more C5− fractions or hydrogenated C5− fractions, such as C4, C5, or a mixed C4/C5 stream, to the fluid catalytic cracking unit and/or to the steam cracker system. As further means, a flow line may be provided for feeding the raffinate fraction to the fluid catalytic cracking unit.

An aromatics processing unit is provided for processing the C6 to C8 fraction produced in the diprolene pyrolysis gasoline unit. The aromatics processing unit may include one or more unit operations, such as an aromatics dealkylation unit, an aromatics extraction unit, and an aromatics saturation unit, and may be configured to produce a non-aromatic hydrocarbon stream. Depending upon the configuration and arrangement of the included unit operations within the aromatics processing unit, embodiments may also produce one or more aromatics products (such as benzene, toluene, and xylenes (BTX)), and one or more cycloalkane products. The resulting streams may then be recovered as a product or may be recovered as a feedstock for further processing in the steam cracker unit or the catalytic cracking unit.

In some embodiments, the aromatics processing unit includes an aromatics saturation unit. The aromatics saturation unit may include one or more reactors for contacting the C6 to C8 cut with hydrogen over an aromatics hydrogenation catalyst to produce a hydrogenated aromatics product containing saturated hydrocarbons, including a mixture of cycloalkanes and C2 to C4 paraffins. The saturated hydrocarbon mixture may then be fed to the steam cracking unit for production of additional ethylene and propylene. In some embodiments, the aromatics saturation unit includes a separation system to separate unreacted hydrogen from the saturated reactor effluent. In various embodiments, the separation system may include separators configured for separating a cycloalkane fraction or a cycloalkane-rich fraction from a C6 to C8 non-aromatics or C2 to C4 paraffin-rich fraction. The C2 to C4 paraffinic fraction may be fed to the steam cracker unit for conversion to ethylene and propylene. Alternatively, the cycloalkanes may be fed to the steam cracker unit or may be fed to the catalytic cracking unit to produce a more suitable non-cyclic hydrocarbon feed that may then be fed to the steam cracker unit.

In some embodiments, the aromatics processing unit includes an aromatics dealkylation unit and an aromatics saturation unit. The aromatics dealkylation unit may include one or more reactors configured to produce a benzene product from an aromatics containing feedstock. For example, toluene and xylenes present in the C6 to C8 fraction may be converted to benzene in the aromatics dealkylation unit. The resulting dealkylation reaction product may then be separated to provide a benzene product stream and a paraffin stream. The paraffin stream may be fed to the steam cracker unit, while the benzene may be recovered as a product, or, depending upon market demand, a portion or an entirety of the benzene produced may be saturated in an aromatics saturation unit to provide a cycloalkane that may be fed to either the steam cracker unit or the hydrocracker unit, as described above.

In some embodiments, the aromatics processing unit includes a BTX extraction unit and an aromatics saturation unit. The BTX extraction unit may include one or more extractive distillation columns, for example, to separate the aromatic compounds from non-aromatic compounds contained in the C6 to C8 fraction. The C6 to C8 non-aromatics may be fed to the steam cracker unit for production of additional ethylene and propylene. The aromatic compounds may be recovered as a BTX product, or, depending upon market demand, a portion or an entirety of the BTX produced may be saturated in an aromatics saturation unit to provide a cycloalkane that may be fed to either the steam cracker unit or the catalytic cracking unit, as described above.

The inclusion of either an aromatics dealkylation unit or a BTX extraction unit with an aromatics separation unit provide flexibility to processes herein to capture aromatics for sale while also providing for conversion of any excess aromatics to propylene and ethylene, accommodating the fluctuations in market demand for aromatics.

Embodiments herein may also include an olefins conversion unit (OCU) to provide additional flexibility to produce additional propylene, again providing flexibility to meet market demand. The olefin conversion unit includes one or more metathesis reaction systems for converting ethylene, received from the product recovery section, and butenes, pentenes, or a mixture thereof, received from the splitter, to produce propylene via metathesis (for example, ethylene+butene→2 propylene, among other possible reactions). In addition to the reactors for performing various metathesis reactions, the olefin conversion unit may include reactors or catalyst zones to isomerize 1-butene or 1-pentene, for example, to form 2-butene or 2-pentene, thus providing additional reactant for the desired production of propylene. Further, the olefin conversion unit may include one or more separators (distillation columns, flash drums, etc., and associated overhead and reboiler systems) for separation and recovery of the propylene. The resulting propylene may then be recovered as a product fraction, while reaction byproduct fractions may be recycled internally within the OCU or may be fed to either or both of the steam cracker system or the catalytic cracking system.

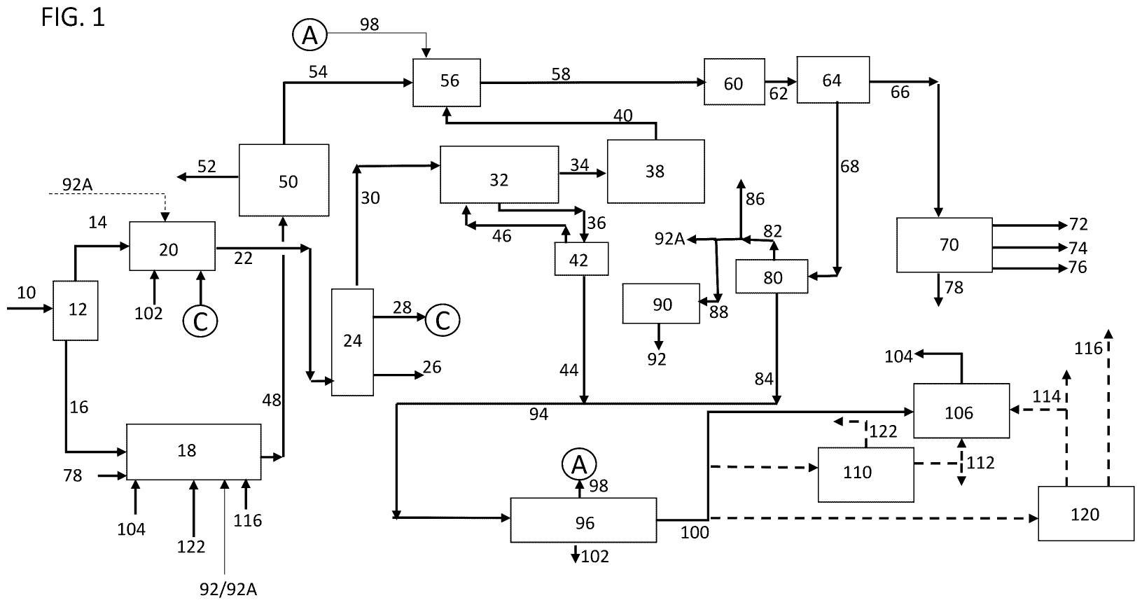

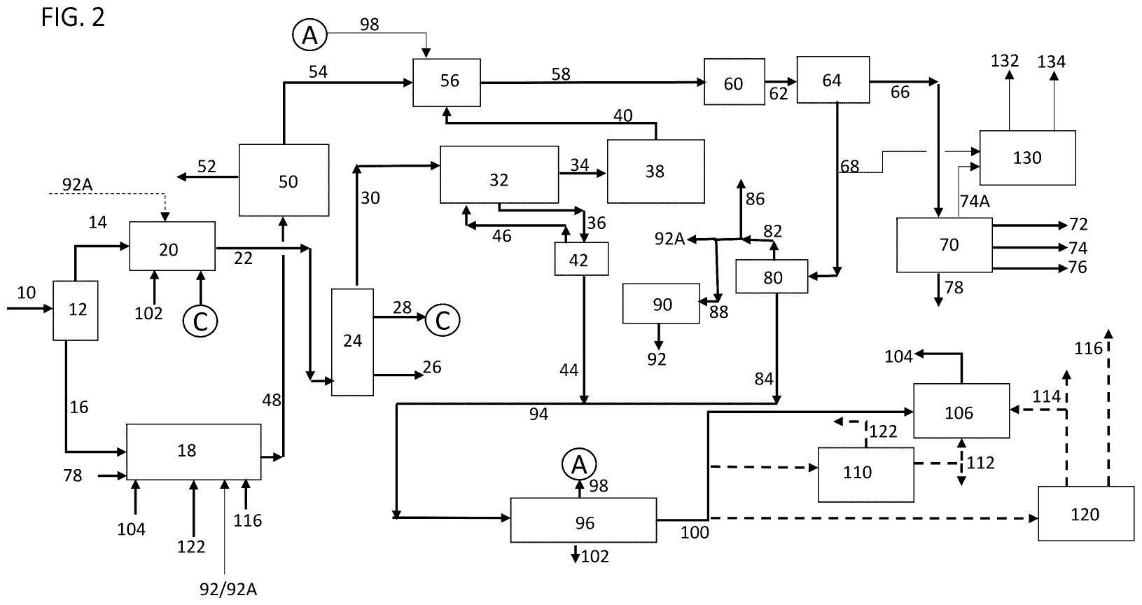

Simplified block process flow diagrams of systems for producing olefins and aromatics according to embodiments herein is illustrated in the accompanying FIG. 1 and FIG. 2, where like numerals represent like parts. Referring now to FIG. 1, a wide boiling hydrocarbon feedstock 10 is fed to a separation system 12 for separating the wide boiling hydrocarbon feedstock into a heavy fraction 14 and a light fraction 16. A fluid catalytic cracking unit 20 catalytically cracks the heavy fraction 14 to produce a catalytically cracked effluent 22. A fractionation system 24 fractionates the catalytically cracked effluent 22 to recover at least a heavy catalytically cracked fraction 26, a naphtha range fraction 28, and a wet gas fraction 30. A flow line may be provided for feeding the naphtha range fraction 28 to the fluid catalytic cracking unit 20 (the flow line illustrated via connector C) to produce additional olefins.

A wet gas compression system 32 compresses and separates the wet gas fraction 30 to form a light compressed fraction 34 and a heavy compressed fraction 36. The light compressed fraction 34 is fed to an impurities removal system 38 for removing impurities from the light compressed fraction, forming a purified light compressed fraction 40. The heavy compressed fraction 36 is fed to a stripper 42 for stripping the heavy compressed fraction to recover a stripped heavy fraction 44 and a stripper gas fraction 46. A flow line or mixing system may be provided for combining the stripper gas fraction 46 with the wet gas fraction 30 upstream of the wet gas compression system 32, thereby maximizing recovery of the heavier hydrocarbons from the wet gas fraction 30.

The light fraction 16 is fed to a steam cracker system 18 for thermally cracking the light fraction to produce a thermally cracked effluent(s) 48. A quench and separation system 50 is then used for quenching and separating the thermally cracked effluent to recover a fuel oil fraction 52 and a light thermally cracked fraction 54.

The light thermally cracked fraction 54 and the purified light compressed fraction 40 are then combined to form a combined catalytic and thermally cracked light fraction. The combined streams are then fed to compression system 56, and the resulting compressed lights stream 58 is fed to drier 60 for drying the combined catalytic and thermally cracked light fraction to form a dried lights fraction 62. Lights separation system 64 then separates the dried lights fraction 62 to recover a C4+ hydrocarbon fraction 68 and a C3− hydrocarbon fraction 66.

Splitter 80 is then used for separating the C4+ fraction 68 into one or more C5-fractions 82 and a C6+ fraction 84. The C6+ fraction 84 is then combined with the stripped heavy fraction 44 recovered from stripper 42 to form a combined C6+ fraction 94 fed to diprolene pyrolysis gas unit 96, which may include a selective hydrogenation system for partially hydrogenating the combined C6+ fraction 94 to form a hydrogenated C6+ fraction and a heavies separation system for separating the hydrogenated C6+ fraction to recover an offgas fraction 98, an aromatics rich naphtha fraction 100, and a raffinate fraction 102. The offgas fraction 98 may be fed to the compression and drying system 56, 60 for recovery of the light olefins therein (connector A). The raffinate fraction 102 may be fed to the fluid catalytic cracking unit 20 for production of additional light olefins.

The C5− fraction(s) 82 may be recovered as product streams 86 or optionally to the fluid catalytic cracking unit 20 (stream 92A) to produce additional olefin product. Alternatively, or additionally, a portion of the C5− fraction(s) 82 may be fed to a hydrogenation system 90, forming paraffinic or isoparaffinic C4 and C5 compounds, which may then be fed via flow line 92 to the steam cracker system 18.

A light olefin product recovery section 70 is provided for separating the C3-fraction 66 to recover a hydrogen-containing offgas fraction 72, an ethylene fraction 74, a propylene fraction 76, and one or more light paraffin fractions 78. The light paraffin fraction(s) 78 may be fed via flow line 78 to the steam cracker system 18 to produce additional olefins.

The C6-C8 cut stream 100 generated from the diprolene pyrolysis gasoline section 96 may be saturated to form cycloalkanes 104 in an aromatics saturation unit 106, which may be cracked in the steam cracker 16 to further improve the light olefins yields.

Alternatively, the C6-C8 cut may be processed in an aromatics dealkylation unit 110 or a BTX extraction unit 120. Benzene 112 or BTX 114 may be recovered as a product or may be further saturated to cycloalkanes in aromatics saturation unit 106 and fed to the steam cracker unit 16 or catalytic cracking unit 20 as needed. For example, C6-C8 non-aromatics 116 from the BTX Extraction Unit 120 may be processed in the steam cracker 16 unit or the catalytic cracking unit 20. Similarly, light paraffins 122 recovered from aromatics dealkylation unit 110 may be processed in the steam cracker 16 unit or the catalytic cracking unit 20.

Referring now to FIG. 2, an olefin conversion unit 130 may be provided for increased propylene production. The olefin conversion unit 130 may receive a portion 74A of ethylene stream 74 recovered in product recovery section 70. The olefin conversion unit 130 may also receive a portion or an entirety of the C4 and/or C5 olefins 86 recovered in separation system 80. The ethylene, butenes, and/or pentenes may then be reacted to form additional propylene 132. Byproducts 134 may also be recovered, and which may be further processed in the catalytic cracking unit 20 or steam cracker 16 to improve light olefins yields.

Various hydrogen supply streams (not illustrated) are supplied to various hydrogenation, isomerization, metathesis, or other reactors within the system. The hydrogen supply streams may be provided by a common feed, purification, compression, and recycle systems in some embodiments. In other embodiments, separate hydrogen feed, purification, compression, and recycle systems may be associated with each unit.

As outlined above, embodiments herein may provide for conversion of crude oils and other wide boiling hydrocarbon mixtures to maximize petrochemical building blocks such as ethylene, propylene and aromatics rich naphtha. Butadiene, butene-1, benzene, toluene, and xylene can also be extracted as separate products utilizing supplemental units.

Embodiments herein may maximize production of light olefins such as ethylene and propylene from direct crude processing by saturating the aromatics generated in the process.

The processing schemes herein involve recovering the raw cuts of the crude and processing the same in FCC and Steam Cracker complexes and finally recovering light olefins (Ethylene and Propylene). The aromatics generated in the process are processed in Diprolene Pyrolysis Gasoline (DPG) followed by Aromatics Saturation Unit (ASU). Saturated aromatics are recycled and cracked in the steam cracker (SC), thus maximizing the total light olefins make in crude to chemicals configuration.

Alternatively, the C6-C8 cut from DPG can be processed in an Aromatics Dealkylation Unit.

Benzene can be recovered as a product or further saturated to cyclohexane in the Aromatics Saturation Unit, recycled, and cracked in the steam cracker. Light Paraffins from Aromatics Dealkylation Unit are also recycled to steam cracker.

Alternatively, the C6-C8 cut from DPG may be processed in a BTX extraction Unit. BTX can be recovered as product or further saturated to Cycloalkanes in the Aromatics Saturation Unit, recycled, and cracked in the steam cracker. C6-C8 Non-aromatics from the BTX Extraction Unit are also processed in the steam cracker.

Embodiments herein may thus be used to meet the increasing demand of light olefins. While steam crackers are renowned for their high ethylene yield with light feedstock processing, FCC processes have emerged as a dominant route for propylene production with heavy feeds. The Pyrotol® and Benzene CD Hydro® technologies may further process C6-C8 cut into Benzene or Cycloalkanes as required. Cycloalkanes can then be processed in the Steam Cracker.

The various technologies are configured in optimum fashion, thereby reducing operational complexities. The goal is to achieve higher yields of olefins while maintaining flexibility in feedstock processing and product demand.

Embodiments herein maximize the conversion of crude oil into light olefins, while providing enhanced flexibility in terms of feed and product processing. By doing so, embodiments herein effectively minimize capital expenditures, maintenance costs, and plot area requirements.

The conventional wisdom directs towards utilizing upgradation of the bottom of the barrel to maximize crude conversion to light olefins. In contrast, embodiments herein utilize the unique combination of FCC, SC and ASU which provides incremental benefits to convert aromatics into light olefins.

Crude to Chemicals (Propylene and Ethylene) conversion is typically 45-55 wt % using integration of steam cracker and FCC/SRDC, however, by integrating units according to embodiments herein, crude conversion is increased to 65-90 wt %.

Another remarkable aspect of embodiments herein is the flexibility in accommodating various feedstocks and adjusting product ratios. If there are changes in the quality of the feedstock, the integrated scheme can easily adapt to handle it. Similarly, if there is a need to modify the Propylene/Ethylene ratio, it can be readily adjusted within the system. The scheme is also flexible with respect to the needs to generate either Benzene product or a mix of Benzene-Toluene-Xylene product or no aromatics at all, giving refiners a flexibility to adjust the operations easily depending on the market needs.

This scheme also provides flexibility processing various feeds from condensates to crude with the end point boiling range up to 550° C. or in some cases up to 650° C. or as high as 700° C. Hydrocarbon mixtures may include whole crudes, virgin crudes, hydroprocessed crudes, gas oils, vacuum gas oils, heating oils, jet fuels, diesels, kerosene, gasolines, synthetic naphthas, raffinate reformates, Fischer-Tropsch liquids, Fischer-Tropsch gases, natural gasolines, distillates, virgin naphthas, natural gas condensates, atmospheric pipestill bottoms, vacuum pipestill stream including bottoms, wide boiling range naphtha to gas oil condensates, heavy non virgin hydrocarbon streams from refineries and plastic pyrolysis oil among others. If desired, these feeds may be pre-processed to remove a portion of sulfur, nitrogen, metals & Conradson Carbon upstream of processes disclosed herein.

When the end boiling point of the hydrocarbon mixture is high, such as above 480-500° C., it cannot be directly processed in the pyrolysis heater to produce olefins, as the heater cokes rapidly. While limiting the reactions conditions may reduce the fouling tendencies, the less severe conditions result in a significant loss in the yields. Whole crude is not cracked as it is not economical due to sub-optimal yields for co-processing of all the different cuts and also because of coking tendencies of heavy tail end present in the crude. It is generally fractionated, and only specific cuts are used in the pyrolysis heater to produce olefins. The remainder is used in the other processes.

In the current schemes, the feed is processed in the Crude Raw Separation Unit which can utilizes the Steam Cracker furnace(s) to preheat and subsequently steam stripping of the crude to separate it at the desired cut point. Steam used for stripping the light end of the crude also serves as a diluent which helps reduce the hydrocarbon partial pressure in coil thus improving the yields further. Lighter cut may be in the boiling range from end point 70° C. to 360° C. or as high as 500° C. and heavier cut boiling range (TBP) may be from 360° C. to >700° C. Based on the quality of crude and the distribution of constituent paraffins, aromatics, naphthenes and olefins, multiple raw separation units can also be utilized in the scheme to generate more than two cuts which are selectively processed in cracking heaters at different optimum point to maximize the olefin yield or can be processed in FCC/SRDC unit or rejected to fuel oil pool.

Heavier Hydrocarbons (C6+) from the Splitter are mixed with FCC stripper bottoms & sent to the Diprolene Pyrolysis Gasoline (DPG) section which hydrogenates the olefinic hydrocarbons. The final products from the DPG units are C6-C8 cut and C9-204° C. cut. C6-C8 cut from DPG unit may be processed to produce Cycloalkanes in the Aromatics saturation unit & C9-204° C. cut can be routed to the FCC/SRDC.

Alternatively, C6-C8 cut from DPG unit can be blended with Gasoline pool.

Alternatively, C6-C8 cut can be processed in the Aromatics Dealkylation Unit to generate Benzene and light paraffins. Benzene may be either withdrawn as a product or converted to cycloalkanes in the Aromatics saturation unit. The light paraffins may be recycled to the steam cracker.

Alternatively, C6-C8 cut may be processed in BTX Extraction unit to recover BTX which may be withdrawn as a product or can be processed to Cycloalkanes in the Aromatics saturation unit while C6-C8 non-aromatics from the unit may be recycled back to the Steam Cracker.

Aromatics Saturation Unit uses reactive distillation to convert C6-C8 aromatics into cycloalkanes.

Cycloalkanes along with balance C6-C8 non-aromatics from the Aromatics Saturation Unit can be recycled back to the Steam Cracker to improve the light olefins yields.

Embodiments herein maximize the production of light olefins by upgrading aromatic rich effluent from steam cracker. Saturating these aromatics followed by steam cracking generates additional light olefins at the expense of Aromatics.

Embodiments herein are also flexible for feed variation and change in product demand (Propylene/Ethylene) & other chemicals. Benzene, Toluene, Xylene can be recovered as produced or consumed inside the unit to generate additional light olefins. Also, Toluene and Xylenes can be converted to Benzene for sales, when needed.

The embodiments herein thus ensures to improve the profit margin per ton of feed charge, enhancing the financial returns for refiners.

The Total Investment Cost (TIC) is significantly reduced in embodiments herein due to the utilization of a lesser number of equipment, optimizing the process units.

The reduced equipment count not only contributes to lower investment costs but also leads to a smaller plot size area requirement, optimizing the efficient use of space.

The schemes herein may give a solution to the refiners who are looking for maximizing petrochemicals such as Ethylene and Propylene directly from crude especially when there is significant volatility in the aromatics market.

Unless defined otherwise, all technical and scientific terms used have the same meaning as commonly understood by one of ordinary skill in the art to which these systems, apparatuses, methods, processes and compositions belong.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to +10%, of up to 5%, of up to 2%, of up to 1%, of up to 0.5%, of up to 0.1%, or up to 0.01%.

As used herein, C2 refers to hydrocarbons having two carbon atoms, such as ethane and ethylene. Similarly, C3 refers to propane and propylene, C4 to various butane, butene, and butadiene compounds, including the various isomers. A carbon number plus would refer to a mixture of hydrocarbons having the generally referenced carbon number or a greater number of carbon atoms (e.g., C3+ refers to a mixture of C3, C4, C5, C6, and additional heavier hydrocarbons as may be present in the process described); similarly, a carbon number minus (e.g., C5−) refers to a mixture of generally hydrocarbons having the referenced carbon number or fewer carbon atoms.

Ranges may be expressed as from about one particular value to about another particular value, inclusive. When such a range is expressed, it is to be understood that another embodiment is from the one particular value to the other particular value, along with all particular values and combinations thereof within the range.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed as new and desired to be protected by Letters Patent is:

1. A process for producing olefins and aromatics from wide boiling hydrocarbon feedstock, the process comprising:
separating the wide boiling hydrocarbon feedstock into a light fraction and a heavy fraction;
catalytically cracking the heavy fraction to produce a catalytically cracked effluent;
fractionating the catalytically cracked effluent to recover at least a wet gas fraction, a naphtha range fraction, and a heavy catalytically cracked fraction;
compressing and separating the wet gas fraction to form a light compressed fraction and a heavy compressed fraction;
removing impurities from the light compressed fraction to form a purified light compressed fraction;
stripping the heavy compressed fraction to recover a stripped heavy fraction and a stripper gas fraction;
combining the stripper gas fraction with the wet gas fraction prior to the compressing and separating step;
thermally cracking the light fraction in a steam cracker unit to produce a thermally cracked effluent;
quenching and separating the thermally cracked effluent to recover a fuel oil fraction and a light thermally cracked fraction;
combining the light thermally cracked fraction with the purified light compressed fraction to form a combined catalytic and thermally cracked light fraction;
drying the combined catalytic and thermally cracked light fraction to form a dried lights fraction;
separating the dried lights fraction to recover a C4+ hydrocarbon fraction and a C3− hydrocarbon fraction;
splitting the C4+ fraction into a one or more C5− fractions and a C6+ fraction;
mixing the C6+ fraction with the stripped heavy fraction to form a combined C6+ fraction;
partially hydrogenating the combined C6+ fraction to form a hydrogenated C6+ fraction;
separating the hydrogenated C6+ fraction to recover an offgas fraction, an aromatics rich naphtha fraction, and a raffinate fraction;
separating the C3− fraction to recover a hydrogen-containing offgas fraction, an ethylene fraction, a propylene fraction, and a light paraffin fraction;
feeding the aromatics rich naphtha fraction to an aromatics processing unit for processing the C6 to C8 fraction in one or more of an aromatics dealkylation unit, an aromatics extraction unit, and an aromatics saturation unit to recover a non-aromatic hydrocarbon stream;
feeding the non-aromatic hydrocarbon stream to the steam cracker unit.

2. The process as claimed in claim 1, further comprising feeding the naphtha range fraction to the catalytically cracking step.

3. The process as claimed in claim 1, further comprising feeding the light paraffin fraction to the thermally cracking step.

4. The process as claimed in claim 1, further comprising feeding a portion of the one or more C5− fractions to the catalytically cracking step.

5. The process as claimed in claim 1, further comprising hydrogenating a portion of the one or more C5− fractions to produce a hydrogenated C5− fraction and feeding hydrogenated C5− fraction to the thermally cracking step.

6. The process as claimed in claim 1, further comprising feeding the raffinate fraction to the catalytically cracking step.

7. The process as claimed in claim 1, further comprising feeding the offgas fraction to the compressing and separating step.

8. The process of claim 1, further comprising feeding a portion of the ethylene fraction and a portion of the one or more C5− fractions to an olefin conversion unit to produce propylene via metathesis.

9. A system for producing olefins and aromatics from a wide boiling hydrocarbon feedstock, the system comprising:
a separation system for separating the wide boiling hydrocarbon feedstock into a light fraction and a heavy fraction;
a fluid catalytic cracking unit for catalytically cracking the heavy fraction to produce a catalytically cracked effluent;
a fractionation system for fractionating the catalytically cracked effluent to recover at least a wet gas fraction, a naphtha range fraction, and a heavy catalytically cracked fraction;
a wet gas compression system for compressing and separating the wet gas fraction to form a light compressed fraction and a heavy compressed fraction;
an impurities removal system for removing impurities from the light compressed fraction to form a purified light compressed fraction;
a stripper for stripping the heavy compressed fraction to recover a stripped heavy fraction and a stripper gas fraction;
a flow line for combining the stripper gas fraction with the wet gas fraction upstream of the wet gas compression system;
a steam cracker system for thermally cracking the light fraction to produce a thermally cracked effluent;
a quench and separation system for quenching and separating the thermally cracked effluent to recover a fuel oil fraction and a light thermally cracked fraction;
a mixer for combining the light thermally cracked fraction with the purified light compressed fraction to form a combined catalytic and thermally cracked light fraction;
a compression and drying system for drying the combined catalytic and thermally cracked light fraction to form a dried lights fraction;
a lights separation system for separating the dried lights fraction to recover a C4+ hydrocarbon fraction and a C3− hydrocarbon fraction;
a splitter for splitting the C4+ fraction into one or more C5− fractions and a C6+ fraction;
a mixer for mixing the C6+ fraction with the stripped heavy fraction to form a combined C6+ fraction;
a hydrogenation system for partially hydrogenating the combined C6+ fraction to form a hydrogenated C6+ fraction;
a heavies separation system for separating the hydrogenated C6+ fraction to recover an offgas fraction, an aromatics rich naphtha fraction, and a raffinate fraction;
a product recovery section for separating the C3− fraction to recover a hydrogen-containing offgas fraction, an ethylene fraction, a propylene fraction, and a light paraffin fraction;
an aromatics processing unit for processing the C6 to C8 fraction in one or more of an aromatics dealkylation unit, an aromatics extraction unit, and an aromatics saturation unit to produce and recover a non-aromatic hydrocarbon stream; and
a flow line for feeding the non-aromatic hydrocarbon stream to the steam cracker system.

10. The system as claimed in claim 9, further comprising a flow line for feeding the naphtha range fraction to the fluid catalytic cracking unit.

11. The system as claimed in claim 9, further comprising a flow line for feeding the light paraffin fraction to the steam cracker system.

12. The system as claimed in claim 9, further comprising feeding a portion of the one or more C5− fractions to the fluid catalytic cracking unit.

13. The system as claimed in claim 9, further comprising feeding a portion of the one or more C5− fractions to the steam cracker system.

14. The system as claimed in claim 13, further comprising a hydrogenation system for hydrogenating the portion of the one or more C5− fractions to produce a hydrogenated C5-fraction fed to the steam cracker system.

15. The system as claimed in claim 9, further comprising feeding the raffinate fraction to the fluid catalytic cracking unit.

16. The system as claimed in claim 9, further comprising a flow line for feeding the offgas fraction to the compression and drying system.

17. The system as claimed in claim 9, further comprising an olefin conversion unit for converting a portion of the ethylene fraction and a portion of the C5− fraction to propylene via metathesis.
